(19) United States
(12) Patent Application Publication
(10) Pub. No.: US 2026/0335198 A1
(43) Pub. Date: Oct. 29, 2026

(54) ADHESIVE UNDERARM ODOR AND MOISTURE CONTROL DEVICE

(71) Applicant: Carla Soto, Hollister, CA (US)

(72) Inventor: Carla Soto, Hollister, CA (US)

(21) Appl. No.: 19/642,945

(22) Filed: Apr. 9, 2026

Related U.S. Application Data
(60) Provisional application No. 63/796,522, filed on Apr. 29, 2025.

(51) Int. Cl. A61K 8/02; A61Q 15/00

(52) U.S. Cl. CPC A61K 8/0208; A61Q 15/00

(57) ABSTRACT

The device is comprised of a body formed from a gel-based material having a deformable and conformable structure adapted to match the anatomical contours of an underarm region. The body comprises an adhesive gel matrix and may further comprise an adhesive layer configured to temporarily secure the body to skin without irritation. The body further comprises functional ingredients including odor-neutralizing agents, antimicrobial compounds, perspiration-reducing agents, and moisture-absorbing materials distributed within the gel matrix to enable controlled release. The device further comprises a removable film releasably coupled to the body to protect the adhesive surface prior to use, wherein the film may include a pull tab to facilitate handling. In operation, the body is applied to the underarm region, wherein adhesion secures placement and enables transfer of functional ingredients over time, followed by removal and disposal or dissolution of the body.

### CROSS-REFERENCE TO RELATED APPLICATION

The present application claims priority to, and the benefit of, U.S. Provisional Application No. 63/796,522, which was filed on Apr. 29, 2025, and is incorporated herein by reference in its entirety.

### FIELD OF THE INVENTION

The present invention relates generally to the field of deodorants and anti-perspirants. More specifically, the present invention relates to a device configured to removably adhere to an underarm region and deliver odor-neutralizing, antimicrobial, and moisture-controlling agents in a controlled manner over time. Accordingly, the present disclosure makes specific reference thereto. Nonetheless, it is to be appreciated that aspects of the present invention are also equally applicable to other like applications, devices, and methods of manufacture.

### BACKGROUND

Individuals frequently leave their homes without applying deodorant, resulting in concern over body odor throughout the day. This issue becomes more pronounced during long work hours, travel, or physical activity where access to hygiene products is limited. Traditional deodorant products are typically bulky and not easily portable in small personal storage spaces. Carrying such items in a purse, backpack, or pocket can be inconvenient and may discourage consistent use. In situations where deodorant is forgotten, individuals may be required to purchase additional products, leading to unnecessary expense. Existing solutions do not adequately address the need for a compact and discreet alternative. Furthermore, conventional deodorants often require manual application methods that are not suitable for quick or private use in all environments. The lack of a convenient, single-use option contributes to inconsistent hygiene maintenance.

Therefore, there exists a long-felt need in the art for a device that enables convenient, on-the-go application of deodorant or anti-perspirant without requiring bulky containers. There also exists a long-felt need in the art for an adhesive underarm odor and moisture control device that provides effective odor and perspiration control through a compact and portable form. Moreover, there exists a long-felt need in the art for an adhesive underarm odor and moisture control device that adheres safely to the skin while delivering active ingredients in a controlled manner.

The subject matter disclosed and claimed herein, in one embodiment thereof, comprises an adhesive underarm odor and moisture control device. The device is comprised of a body formed from a gel-based material having a deformable and conformable structure that can be adapted to match the anatomical contours of an underarm region. The body comprises an adhesive gel matrix and may further comprise an adhesive layer configured to temporarily secure the body to skin without irritation. The body further comprises functional ingredients including odor-neutralizing agents, antimicrobial compounds, perspiration-reducing agents, and moisture-absorbing materials distributed within the gel matrix in homogeneous or structured configurations to enable controlled release. The body may further comprise a permeability structure including micro perforations or breathable channels configured to permit airflow and moisture vapor transmission. A surface texture may be integrated into the body to regulate transfer rates and enhance adhesion. The device further comprises a removable film releasably coupled to the body to protect the adhesive surface prior to use, wherein the film may include a pull tab to facilitate handling. In operation, the body is applied to the underarm region of a user, wherein adhesion secures placement and enables transfer of functional ingredients over a duration of time, followed by removal and disposal or dissolution of the body.

In this manner, the adhesive underarm odor and moisture control device of the present invention accomplishes all the foregoing objectives and provides a compact and portable solution that eliminates the need for bulky deodorant containers and enables discreet, on-the-go application. More specifically, the conformable gel-based body and adhesive properties ensure secure placement onto a user's skin and ease of use in various environments. The incorporation of functional ingredients within the body matrix further allows for effective odor neutralization, antimicrobial action, and moisture control over a sustained period. Additionally, the removable film and packaging enhance convenience, hygiene, and product stability. Consequently, the invention addresses the limitations of traditional deodorant and anti-perspirant products while offering an efficient and user-friendly alternative for maintaining personal hygiene.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an adhesive underarm odor and moisture control device. The device is a single-use underarm odor and moisture control device configured to apply odor-controlling and perspiration-reducing compounds without stick, roll-on, or spray applicators. The device is suitable for travel, commuting, athletic activity, occupational settings, and social use, and is dimensioned for storage in compact personal locations to enhance portability and accessibility.

The device is comprised of a body formed from a gel-based material having a flexible, deformable, compressible, and conformable consistency for adaptation to underarm contours. In one embodiment, the body may be comprised of a surface texture to improve adhesion, regulate ingredient transfer, and enhance tactile feedback. The surface texture may include microchannels configured to direct movement of released ingredients across the skin. The body may also be comprised of a permeability structure to permit air exchange and moisture vapor transmission, thereby improving comfort and reducing irritation during wear.

The body may be comprised of an adhesive gel matrix enabling temporary skin adherence, optionally supplemented by an adhesive layer positioned on one or more surfaces in one embodiment. The adhesive layer is configured to retain placement during movement while allowing painless removal with minimal residue.

The body may be comprised of functional ingredients providing odor control, antimicrobial action, perspiration reduction, and moisture absorption. The functional ingredients may be distributed in various configurations and incorporated in forms enabling controlled release mechanisms including time, pressure, temperature, or moisture activation. The functional ingredients may include odor-neutralizing agents, antimicrobial compounds, sweat-reducing salts, and moisture-absorbing materials to provide hygiene and moisture management. The body may be comprised of secondary ingredients supporting structure, sensory characteristics, and stability, including fragrances, carrier materials, stabilizers, preservatives, and botanical extracts providing conditioning effects.

The device may be comprised of a removable film serving as a protective and handling layer to prevent premature exposure of the adhesive and ingredients. The film may include a pull tab. The film and body may be stored within a package formed as a pouch or container providing resistance to environmental factors, and the package may include a closure fastener enabling resealable access in multi-use embodiments. During use, the film is removed from the package and separated from the body prior to or after application. The body is applied to an underarm region and adheres to the skin, remaining in place for a duration during which functional ingredients transfer. The body is subsequently removed and discarded or allowed to dissolve depending on embodiment.

A first method comprises providing the device with the body, film, and package; opening the package; removing the film from the body; separating the body from the film; pressing the body onto an underarm region to adhere and conform; maintaining contact for ingredient transfer; and removing or dissolving the body after use.

A second method comprises providing the device with the body, film, and package; opening the package; removing the film from the body; pressing the body onto an underarm region while coupled to the film; peeling the film away after adhesion; maintaining contact for ingredient transfer; and removing or dissolving the body after use.

Accordingly, the adhesive underarm odor and moisture control device of the present invention is particularly advantageous as it provides a compact and portable solution that eliminates the need for bulky deodorant containers and enables discreet, on-the-go application. More specifically, the conformable gel-based body and adhesive properties ensure secure placement onto a user's skin and ease of use in various environments. The incorporation of functional ingredients within the body matrix further allows for effective odor neutralization, antimicrobial action, and moisture control over a sustained period. Additionally, the removable film and packaging enhance convenience, hygiene, and product stability. Consequently, the invention overcomes the limitations of traditional deodorant and anti-perspirant products while offering an efficient and user-friendly alternative for maintaining personal hygiene.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of an adhesive underarm odor and moisture control device of the present invention while secured to an underarm area of the user in accordance with the disclosed architecture;

FIG. 2 illustrates a perspective view of one potential embodiment of an adhesive underarm odor and moisture control device of the present invention while the body is being removed from the film in accordance with the disclosed architecture;

FIG. 3 illustrates a perspective view of one potential embodiment of an adhesive underarm odor and moisture control device of the present invention while the body is secured on the film within the package in accordance with the disclosed architecture;

FIG. 4 illustrates a flowchart of a method of using one potential embodiment of an adhesive underarm odor and moisture control device of the present invention in accordance with the disclosed architecture; and

FIG. 5 illustrates a flowchart of a method of using one potential embodiment of an adhesive underarm odor and moisture control device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a device that enables convenient, on-the-go application of deodorant or anti-perspirant without requiring bulky containers. There also exists a long-felt need in the art for an adhesive underarm odor and moisture control device that provides effective odor and perspiration control through a compact and portable form. Moreover, there exists a long-felt need in the art for an adhesive underarm odor and moisture control device that adheres safely to the skin while delivering active ingredients in a controlled manner.

The present invention, in one exemplary embodiment, is comprised of an adhesive underarm odor and moisture control device. The device is a single-use underarm odor and moisture control device configured to deliver odor-controlling and perspiration-reducing compounds without stick, roll-on, or spray applicators. The device is suitable for portable use across various environments and is dimensioned for compact storage.

The device is comprised of a body formed from a gel-based material having a flexible, deformable, and conformable structure for adaptation to underarm contours. The body may be comprised of a surface texture to enhance adhesion and regulate ingredient transfer and may include microchannels for directing ingredient movement. The body may further be comprised of a permeability structure permitting air and moisture vapor transmission to improve comfort.

The body may be comprised of an adhesive gel matrix enabling temporary adherence to skin, optionally supplemented by an adhesive layer configured to maintain placement while allowing painless removal with minimal residue. The body may be comprised of functional ingredients providing odor control, antimicrobial action, perspiration reduction, and moisture absorption, wherein the functional ingredients are arranged to enable controlled release. The body may further be comprised of secondary ingredients supporting structure, stability, and sensory characteristics.

The device may be comprised of a removable film serving as a protective layer, and the film and body may be stored within a package having a closure fastener. During use, the body is applied to an underarm region, remains in place for ingredient transfer, and is subsequently removed or dissolved.

A first method comprises providing the device, opening the package, removing the film and body, separating the body, applying the body to an underarm region, maintaining contact for transfer, and removing or dissolving the body. A second method comprises providing the device, opening the package, removing the film and body, applying the body while coupled to the film, removing the film after adhesion, maintaining contact for transfer, and removing or dissolving the body.

As such, the device provides a compact and portable solution enabling discreet application, wherein the gel-based body and adhesive properties support secure placement and effective odor, microbial, and moisture control.

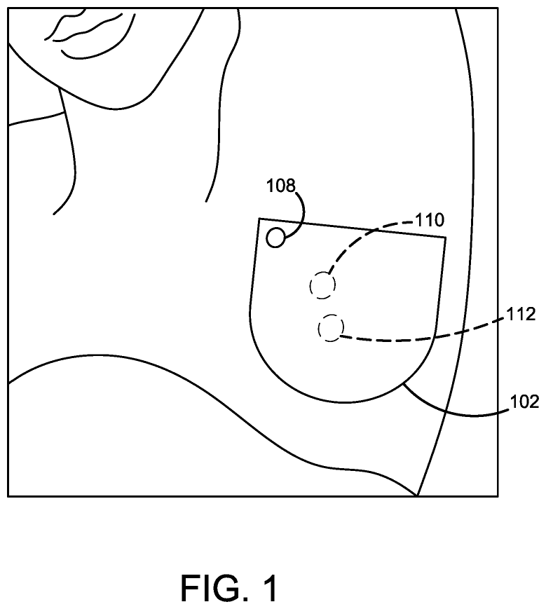

Referring initially to the drawings, FIG. 1 illustrates a perspective view of one potential embodiment of an adhesive underarm odor and moisture control device 100 of the present invention while secured to an underarm area of the user in accordance with the disclosed architecture. The device 100 is a single-use underarm odor and moisture control device that enables a user to rapidly apply odor-controlling and/or perspiration-reducing compounds to an underarm region without reliance on traditional stick, roll-on, or spray applicators. As such, the device 100 may be utilized in a wide range of environments such as but not limited to travel, commuting, athletic activity, occupational settings, social interactions, etc., wherein discreet and efficient hygiene maintenance may be desirable. The device 100 may be dimensioned and configured to enable storage in compact personal storage locations such as but not limited to a purse, backpack, pocket, wallet compartment, athletic bag, glove compartment, etc., thereby facilitating portability and accessibility.

The device 100 is comprised of a body 102. The body 102 may be made from a gel-based material having a gelatin-like consistency that may be flexible, deformable, compressible, and conformable to the anatomical contours of a user's underarm region. The body 102 may be formed in a variety of geometries such as but not limited to oval, circular, crescent-shaped, contoured, tapered, winged, rectangular, asymmetrical, ergonomically sculpted, etc., to enhance contact surface area and ensure uniform application. The body 102 may vary in thickness along one or more regions in one embodiment, wherein for example a central region may be thicker for increased ingredient capacity and peripheral regions may be thinner to improve edge conformity and reduce detachment likelihood of the body 102 from a user's skin.

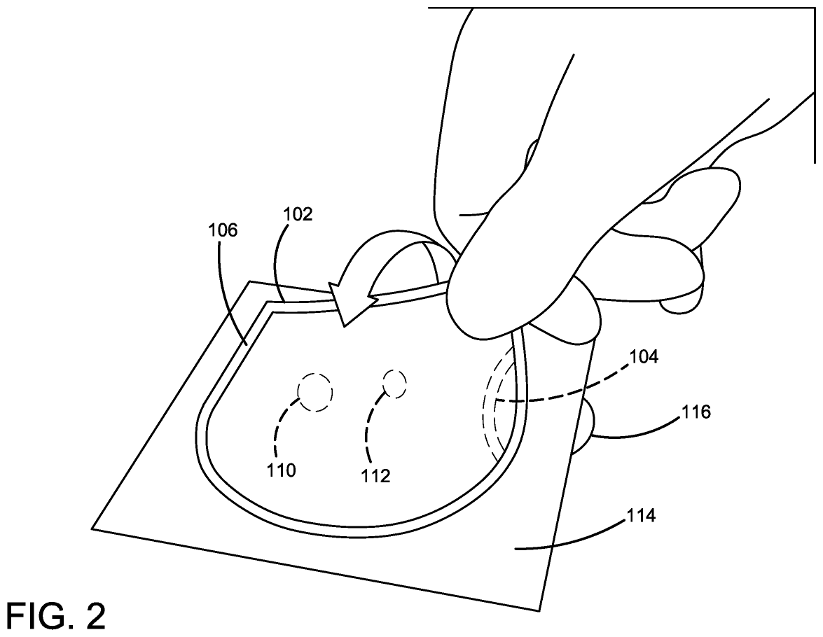

In one embodiment, the body 102 may further be comprised of a surface texture 104 (as seen in FIG. 2) such as ridged, micro-patterned, lattice-structured, embossed, dimpled, etc., wherein such surface texture 104 may improve adhesion, regulate transfer rates of active ingredients, and enhance tactile feedback during application and use of the body 102. In some embodiments, the surface texture 104 may be in the form of microchannels that may be configured to direct flow of liquefied or pressure-released ingredients across the skin surface.

The body 102 material may be in the form of an adhesive gel matrix that enables temporary adherence to a user's skin (as seen in FIG. 1) without irritation. The adhesive properties of the body 102 may be intrinsic to the gel composition and/or supplemented by an adhesive layer 106 that may be positioned on one or more surfaces of the body 102. The adhesive layer 106 may be comprised of materials such as but not limited to pressure-sensitive adhesives, hydrogel adhesives, silicone-based adhesives, acrylic adhesives, bio-adhesive polymers, etc. The adhesive strength of the body 102 and/or layer 106 may be configured to provide sufficient retention on the user's skin during movement while enabling painless removal and minimal residue.

The body 102 may further be comprised of a permeability structure 108, as seen in FIG. 1. The permeability structure 108 may include semi-permeable regions, micro perforations, breathable channels, etc., that may allow air exchange and moisture vapor transmission between the skin of the user and the ambient environment. Such permeability may reduce skin irritation and improve user comfort during extended wear.

The body 102 may further be comprised of functional ingredients 110 that may provide odor control, antimicrobial action, perspiration reduction, and/or moisture absorption. The functional ingredients 110 may be distributed in a variety of configurations such as but not limited to homogeneous distribution, gradient distribution, discrete zones, layers, etc. In one embodiment, the functional ingredients 110 may be incorporated in forms such as but not limited to dissolved phases, suspended particulates, microcapsules, nano capsules, emulsions, phase-separated domains, interpenetrating polymer networks, etc., to enable controlled release mechanisms such as time-release, pressure-activated release, temperature-activated release, moisture-triggered release, etc.

The functional ingredients 110 may be in the form of odor-neutralizing agents such as but not limited to zinc compounds, cyclodextrins, sodium bicarbonate, activated charcoal, enzymatic neutralizers, mineral-based absorbers, etc. The functional ingredients 110 may also be in the form of antimicrobial agents such as but not limited to alcohol-based compounds, silver-based materials, plant-derived antimicrobials, organic acids, peptides, bacteriostatic compounds, etc. The functional ingredients 110 may further be sweat-reducing compounds such as but not limited to aluminum chlorohydrate, aluminum zirconium tetrachlorohydrex gly, aluminum sesquichlorohydrate, and related salts. Additionally, the functional ingredients 110 may be moisture-absorbing materials such as but not limited to silica, starch derivatives, modified cellulose, superabsorbent polymers, clay minerals, etc.

The body 102 may further be comprised of secondary ingredients 112 that may support structural integrity, sensory experience, and/or product stability. The secondary ingredients 112 may be in the form of encapsulated fragrances, essential oils, synthetic aromatic compounds, etc., wherein release may be continuous, delayed, or conditionally triggered during use. The secondary ingredients 112 may also be in the form of carrier materials such as but not limited to water, glycerin, propylene glycol, polyethylene glycol, hydrocolloids, elastomeric gels, crosslinked polymer matrices, etc. Stabilizers and preservatives such as but not limited to antioxidants, chelating agents, organic acids, phenoxyethanol, etc., may also be secondary ingredients 112 that are incorporated to maintain chemical integrity. Other ingredients 112 can include botanical extracts such as but not limited to aloe vera, chamomile, calendula, green tea, witch hazel, etc., which may provide soothing and conditioning effects.

The device 100 may also be comprised of a thin film 114 that may serve as a protective and handling layer. The film 114 may be releasably coupled to the body 102 and may prevent premature exposure of the adhesive body 102 and/or layer 106 and ingredients 110,112. The film 114 may be comprised of materials such as but not limited to polyethylene, polypropylene, polyester, biodegradable films, coated papers, etc., and may be transparent, translucent, or opaque. In one embodiment, the film 114 may include a pull tab 116 (as seen in FIG. 2) to facilitate removal from the body 102 in one embodiment.

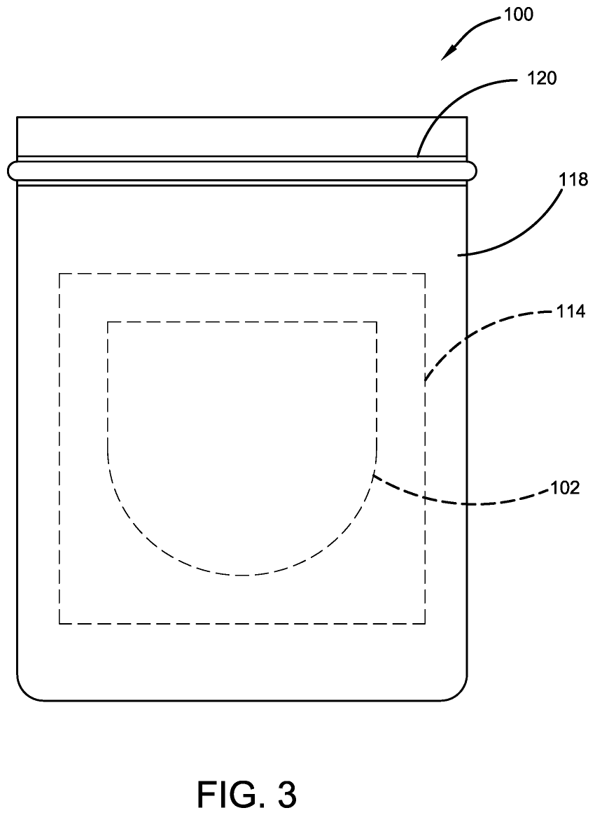

The film 114 and body 102 (while attached to the film 114) may be stored within a package 118, as seen in FIG. 3. The package 118 may be in the form of a flexible pouch, rigid container, etc. The package 118 may be made from materials that may provide resistance to moisture, oxygen, light, and temperature variation. The package 118 may include a closure fastener 120 such as but not limited to a resealable adhesive strip, zipper seal, snap closure, interlocking track, fold-over flap, etc., enabling repeated access in multi-use embodiments.

In use, a user may remove the film 114 from the package 118 and separate the body 102 from the film 114 prior to application. Alternatively, the body 102 may be applied to the underarm region while coupled to the film 114, followed by removal of the film 114 after placement. The body 102 may adhere to the skin via inherent adhesive properties and/or the adhesive layer 106. The body 102 may remain in place for a predetermined duration during which functional ingredients 110 may transfer to the skin. The body 102 may subsequently be removed and discarded, or in certain embodiments, the body 102 may partially or fully dissolve during use.

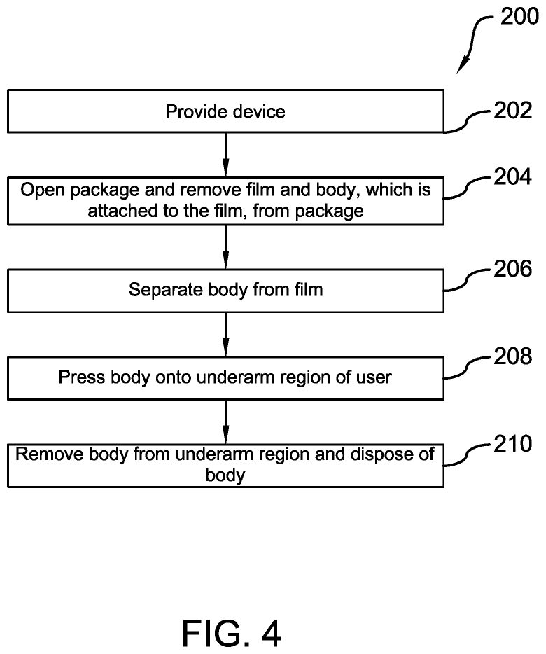

The present invention is also comprised of a first method 200 of using the device 100, as seen in FIG. 4. First, a device 100 is provided comprised of a body 102 comprised of a gel-based material, the material being embedded with functional ingredients 110 and secondary ingredients 112, and a film 114, wherein the film 114 may be stored with the body 102 in a package 118 having a closure fastener 120 [Step 202]. Then, the package 118 may be opened via the closure fastener 120 and the film 114 with the body 102 may be removed from the package 118 [Step 204]. Next, the body 102 may be separated from the film 114 prior to application on a user's skin [Step 206]. Then, the body 102 may be pressed onto an underarm region of a user such that the inherent adhesive properties of the body 102 material (and/or an adhesive layer 106 of the body 102, if present) secure the body 102 to the skin and conform to anatomical contours of the user's underarm area [Step 208]. The body 102 may remain in contact with the underarm region for a duration of time wherein functional ingredients 110 are transferred to the skin. Finally, the body 102 may be removed from the underarm region after use and discarded or alternatively allowed to partially or fully dissolve during use depending on embodiment [Step 210].

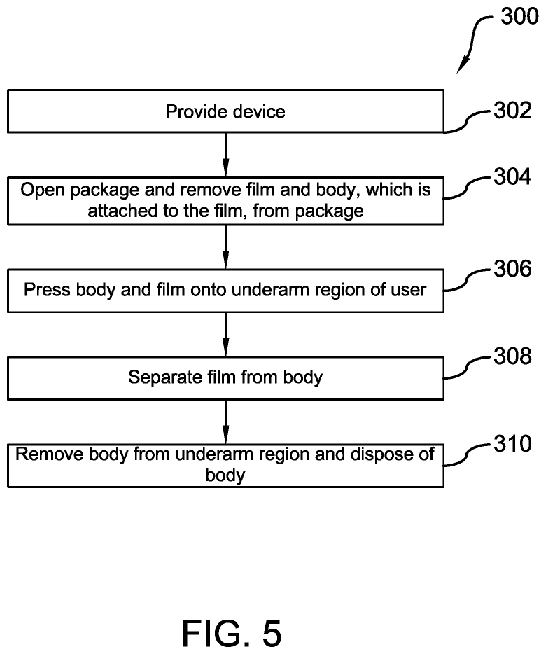

The present invention is also comprised of a second method 300 of using the device 100, as seen in FIG. 5. First, a device 100 is provided comprised of a body 102 comprised of a gel-based material, the material being embedded with functional ingredients 110 and secondary ingredients 112, and a film 114, wherein the film 114 may be stored with the body 102 in a package 118 having a closure fastener 120 [Step 302]. Then, the package 118 may be opened via the closure fastener 120 and the film 114 with the body 102 may be removed from the package 118 [Step 304]. Then, the body 102 may be pressed onto an underarm region of a user such that the inherent adhesive properties of the body 102 material (and/or an adhesive layer 106 of the body 102, if present) secure the body 102 to the skin and conform to anatomical contours of the user's underarm area [Step 306]. Next, the film 114 may be peeled away from the body 102 after the body 102 is secured to the underarm region [Step 308]. The body 102 may remain in contact with the underarm region for a duration of time wherein functional ingredients 110 are transferred to the skin. Finally, the body 102 may be removed from the underarm region after use and discarded or alternatively allowed to partially or fully dissolve during use depending on embodiment [Step 310].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “adhesive underarm odor and moisture control device” and “device” are interchangeable and refer to the adhesive underarm odor and moisture control device 100 of the present invention.

Notwithstanding the foregoing, the adhesive underarm odor and moisture control device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the adhesive underarm odor and moisture control device 100 as shown in the FIGS. are for illustrative purposes only, and that many other sizes and shapes of the adhesive underarm odor and moisture control device 100 are well within the scope of the present disclosure. Although the dimensions of the adhesive underarm odor and moisture control device 100 are important design parameters for user convenience, the adhesive underarm odor and moisture control device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An adhesive underarm odor and moisture control device comprising:
a flexible body comprised of a gel-based material, wherein the gel-based material further comprises a functional ingredient configured to provide odor control, antimicrobial action, perspiration reduction, or moisture absorption; and
a film.

2. The adhesive underarm odor and moisture control device of claim 1, wherein the body further comprises an adhesive layer.

3. The adhesive underarm odor and moisture control device of claim 1, wherein the body further comprises a surface texture.

4. The adhesive underarm odor and moisture control device of claim 1, wherein the body further comprises a permeability structure.

5. The adhesive underarm odor and moisture control device of claim 1, wherein the body has a variable thickness.

6. The adhesive underarm odor and moisture control device of claim 1, wherein the film further comprises a pull tab.

7. The adhesive underarm odor and moisture control device of claim 1, wherein the gel-based material is an adhesive gel matrix.

8. The adhesive underarm odor and moisture control device of claim 1, wherein the gel-based material further comprises a secondary ingredient.

9. The adhesive underarm odor and moisture control device of claim 8, wherein the secondary ingredient is a carrier material.

10. The adhesive underarm odor and moisture control device of claim 8, wherein the secondary ingredient is a stabilizer or a preservative.

11. The adhesive underarm odor and moisture control device of claim 8, wherein the secondary ingredient is a botanical extract.

12. The adhesive underarm odor and moisture control device of claim 8 further comprising a packaging.

13. The adhesive underarm odor and moisture control device of claim 12, wherein the packaging further comprises a closure fastener.

14. The adhesive underarm odor and moisture control device of claim 12, wherein the flexible body is ovular in shape.

15. The adhesive underarm odor and moisture control device of claim 12, wherein the flexible body is contoured in shape.

16. The adhesive underarm odor and moisture control device of claim 8, wherein the flexible body is positioned on the film.

17. The adhesive underarm odor and moisture control device of claim 16, wherein the removable body is removably attached to the film.

18. The adhesive underarm odor and moisture control device of claim 8, wherein the gel-based material is compressible.

19. A method of using an adhesive underarm odor and moisture control device, the method comprising the following steps:
providing an adhesive underarm odor and moisture control device comprised of a body made from a gel-based material embedded with a functional ingredient and a secondary ingredient, and a film, wherein the body is stored on the film;
separating the body from the film;
pressing the body onto an underarm region of a user such that the body adheres to the underarm region;
maintaining the body in contact with the underarm region for a duration of time to transfer the functional ingredients to the underarm region; and
removing the body from the underarm region after a period of time and further disposing of the body.

20. A method of using an adhesive underarm odor and moisture control device, the method comprising the following steps:
providing an adhesive underarm odor and moisture control device comprised of a body made from a gel-based material embedded with a functional ingredient and a secondary ingredient, and a film, wherein the body is stored on the film;
pressing the body onto an underarm region of a user such that the body adheres to the underarm region;
separating the film from the body;
maintaining the body in contact with the underarm region for a duration of time to transfer the functional ingredients to the underarm region; and
removing the body from the underarm region after a period of time and further disposing of the body.
